(19) United States
(12) Patent Application Publication
(10) Pub. No.: US 2009/0093837 A1
(43) Pub. Date: Apr. 9, 2009

(54) ANASTOMOSIS PLUG FOR BARIATRIC SURGERY

(75) Inventor: Travis E. Dillon, Winston-Salem, NC (US)

Correspondence Address: BRINKS HOFER GILSON & LIONE/CHICAGO/COOK, PO BOX 10395, CHICAGO, IL, 60610, US

(73) Assignee: WILSON-COOK MEDICAL INC., Winston-Salem, NC (US)

(21) Appl. No.: 12/247,786

(22) Filed: Oct. 8, 2008

Related U.S. Application Data
(60) Provisional application No. 60/978,753, filed on Oct. 9, 2007.

(51) Int. Cl. A61B 17/12

(52) U.S. Cl. 606/191

(57) ABSTRACT

Medical devices and methods for controlling the flow of contents through an opening, such as an anastomosis formed during bariatric surgery. Generally, the medical device includes a tube and first and second radially extendable anchors attached to the tube. The anchors keep the medical device centered at the anastomosis or other opening, while the inner diameter of the tube restricts the passage of contents therethrough. A limiter may also be employed to vary the smallest diameter of the tube's lumen.

### CROSS-REFERENCE TO RELATED APPLICATIONS

This application claims the benefit of U.S. Provisional Application Ser. No. 60/978,753 filed on Oct. 9, 2007, entitled “ANASTOMOSIS PLUG FOR BARIATRIC SURGERY”

### FIELD OF THE INVENTION

This invention relates to medical devices, and more particularly to devices that can be placed in an anastomosis, such as one formed during bariatric surgery to control gastric emptying.

### BACKGROUND OF THE INVENTION

It is well known that obesity is a very difficult condition to treat. Methods of treatment are varied, and include drugs, behavior therapy, and physical exercise, or often a combinational approach involving two or more of these methods. Unfortunately, results are seldom long term, with many patients eventually returning to their original weight over time. For that reason, obesity, particularly morbid obesity, is often considered an incurable condition. More invasive approaches have been available which have yielded good results in many patients. These include surgical options such as bypass operations or gastroplasty which revise the size of the stomach, often leaving only the fundus or a reduced gastric pouch that leads directly to a portion of the small intestines.

Unfortunately, the revised gastric pouches and the connection to the small intestines tend to adjust and sometimes expand over time, resulting in weight regain after a few years. While a second reduction procedure can be performed, the complication rates are much higher than the original procedure, and the amount of reduction necessary to achieve weight loss results is unknown.

### BRIEF SUMMARY OF THE INVENTION

The present invention provides medical devices and methods for controlling the flow of contents through an anastomosis or other opening between two bodily structures. The medical devices are well tolerated while also being easy to place and retrieve. One embodiment of a medical device, constructed in accordance with the teachings of the present invention, generally includes a tube and first and second radially extendable anchors attached to the tube. The tube defines a lumen extending therethrough through which the contents can flow. The first radially extendable anchor is attached to a proximal portion of the tube, while the second radially extendable anchor is attached to a distal portion of the tube. The medial device is operable between a delivery configuration and a deployed configuration. The first and second anchors move radially outwardly between the delivery configuration and the deployed configuration. In this manner, the anchors keep the medical device centered at the anastomosis or other opening, while the inner diameter of the tube restricts the passage of contents through the anastomosis or other opening.

According to more detailed aspects of the medical devices, the first and second radially extendable anchors may take many forms, such as expandable balloons, a plurality of struts, expandable wire baskets, expandable stents, and Malecot-type catheters having expandable folds. When the first and second anchors are expandable balloons, the tube defines inflation passageways in communication with the expandable balloons. When the first and second anchors comprise a plurality of struts, the struts can have many different constructions and take various shapes. By way of example, the struts may be deflectable and biased towards the deployed configuration, and may also be formed of a shaped memory material and structured to transition from the delivery configuration to the deployed configuration at about body temperature.

According to further detailed aspects of the medical devices, the devices also preferably include a limiter attached to the tube for varying the diameter of at least a portion of the lumen of the tube. The limiter may take many forms, such as a band positioned along the tube which is sized to restrict the diameter, an expandable balloon that is positioned within the lumen, or various other inserts which may be placed within the lumen of the tube to vary the diameter thereof.

Another embodiment of the present invention provides a method for controlling the flow of contents through an opening in tissue, such as an anastomosis. The method includes providing a medical device such as one of the devices described above having a tube and first and second radially extendable anchors. The medical device is delivered to a location proximate the opening in its delivery configuration. The medical device is positioned such that the first anchor is on a proximal side of the opening and the second anchor is on a distal side of the opening. The medical device is operated to move the first anchor radially outwardly to its deployed configuration, and is also operated to move the second anchor radially outwardly towards the deployed configuration.

According to more detailed aspects of the methods, the step of operating the medical device includes inflating the anchors when they are formed as expandable balloons. When the first and second anchors comprise a plurality of struts, the medical device may further include a delivery sheath enclosing the plurality of struts, and the operating steps include relative translation of the delivery sheath and medical device to expose the plurality of struts. The step of operating the first radially expandable anchor may be performed prior to the step of positioning the medical device within the anastomosis, to aid in the positioning. When the medical device includes a limiter, the method may further comprise the step of operating the limiter to adjust the diameter of the tube's lumen. Preferably, the limiter may be adjusted in situ, after the medical device has been placed.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a cross-sectional view of a medical device constructed in accordance with the teachings of the present invention, shown in a delivery configuration.

FIG. 2 is a cross-sectional view taken about the line 2-2 of FIG. 1;

FIG. 3 is a cross-sectional view of the medical device depicted in FIG. 1, shown in its deployed configuration;

FIG. 3A is a enlarged cross-sectional view a portion of the medical device depicted in FIG. 3;

FIG. 4 is a cross-sectional view showing deployment of the medical device within an anastomosis formed between the stomach and small intestines;

FIG. 5 is a cross-sectional view of another embodiment of the medical device constructed in accordance with the teachings of the present invention;

FIG. 6 is a cross-sectional view taken about the line 6-6 in FIG. 5; and

FIG. 7 is a cross-sectional view of the medical device depicted in FIG. 5, showing the device in a delivery configuration.

### DETAILED DESCRIPTION OF THE INVENTION

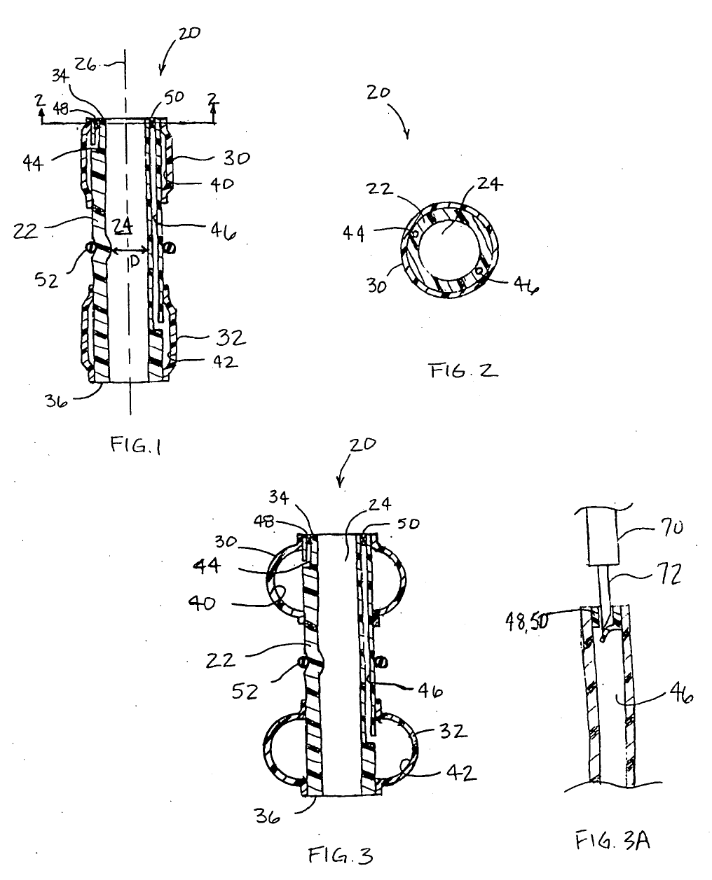

Turning now to the figures, FIGS. 1-3 depict one embodiment of a medical device 20 for controlling the flow of contents through an opening in tissue, constructed in accordance with the teachings of the present invention. The medical device 20 generally includes a tube 22 defining a lumen 24 extending therethrough. The tube 22 and lumen 24 define a longitudinal axis 26 of the medical device 20. A first radially extendable anchor 30 is attached to a proximal portion of the tube 22 while a second radially extendable anchor 32 is attached to a distal portion of the tube 22. More particularly, the first and second anchors 30, 32 are longitudinally spaced apart, and the first anchor 30 is attached near a proximal end 34 of the tube 22, while the second anchor 32 is attached near a distal end 36 of the tube 22.

In the embodiment of FIGS. 1-3, the first and second radially extendable anchors 30, 32 are formed as expandable balloons. In particular, the balloons 30, 32 define inflation chambers 40, 42 that are structured to receive a fluid (i.e. a gas or a liquid such as air, water, saline solution, etc.). The balloons 30, 32 may be either compliant or non-compliant balloons. The inflation chambers 40, 42 are in communication with inflation passageways 44, 46, respectively. The inflation passageways 44, 46 are formed in the tube 22 and extend to the proximal end 34 of the tube 22. At the proximal end 34, valves 48, 50 are positioned to control the passage of the fluids through the inflation passageways 44, 46, to and from the inflation chambers 40, 42.

Accordingly, the medical device 20 and its expandable balloons 30, 32 are operable between a delivery configuration (shown in FIG. 1) and a deployed configuration (shown in FIG. 3). As will be discussed in further detail hereinbelow, the expandable balloons 30, 32 keep the medical device 20 centered within an anastomosis or other opening, while the diameter of the tube lumen 24 restricts the passage of contents through the anastomosis or other opening.

Many well-known plastics have suitable properties for forming the medical device 20 (including the tube 22 and balloons 30, 32) including polyesters, polyurethanes, polyethylenes, polyamides, silicone or other possible materials. Preferably, the medical device 20 is digestive-resistant, meaning the material is not subject to the degradive affects of stomach acid and enzymes, or the general environment found within the gastric system over an extended period of time. This allows the device to remain intact for the intended life of the device. This does not necessarily mean that the material cannot be degraded over time; however, one skilled in the medial arts and gastrological devices would readily appreciate the range of material that would be suitable for use as a long-term intragastric member.

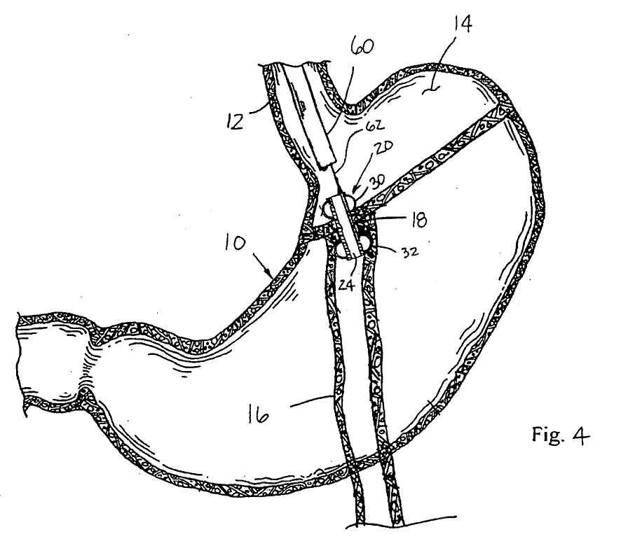

The tube 22 and lumen 24 of the medical device are preferably sized to provide a desired amount or flow rate of contents through the anastomosis 18 (FIG. 4), which can vary from patient to patient. The balloons 30, 32 are preferably about 25 mm or greater in diameter, and are preferably spaced apart about 2 mm to 30 mm, and most preferably about 5 mm to 15 mm. An optional feature is to include a limiter to further control the passage of contents through the tube 22 and its lumen 24. Although the limiter can take various forms, the limiter generally varies the diameter of the tube lumen 24 to control flow rates therethrough. As best seen in FIGS. 1 and 3, an elastic band 52 has been positioned along the tube 22 and around the exterior thereof. The band 52 is preferably formed of a rubber or elastomer (and can include reinforcing elements), and is sized and structured to deform the tube 22 and reduce the inner diameter of the tube 22 and lumen 24. As such, differently sized bands 52 may be utilized to control the smallest inner diameter D, and hence control the flow rate of contents through the medical device 20. The bands 52 should be sized and structured so as to permit the passage of fluids through the inflation passageway 46 for continued operation of the second expandable balloon 32.

It will be recognized by those skilled in the art that numerous different types of limiters may be employed, including adjustable bands, rings, washers, or annular inserts. Expandable devices, such as balloons, folds (e.g. Malecot-type) struts, wire baskets, and stents may also be used to form the limiter. The limiters may be employed along the exterior of the tube 22 as described above, or the limiters may be positioned within the tube 22 for varying the smallest diameter D of the tube lumen 24.

Deployment of the medical device 20 has been depicted in FIG. 4. As shown, the stomach 10 has been modified through bariatric surgery to form a gastric pouch 14 in the area of the stomach's fundus, the gastric pouch 14 receiving contents from the esophagus 12. Further, the small intestine 16 has been liberated and brought into communication with the gastric pouch 14. An anastomosis 18 provides communication between the gastric pouch 14 and the small intestine 16. The medical device 20 is placed within the anastomosis 18 for controlling the flow of contents through the anastomosis 18. In accordance with a method for controlling the flow of contents through the anastomosis 18, the medical device 20 is provided, and delivered in its delivery configuration (FIG. 1) to a location proximate the anastomosis 18. In particular, an endoscope 60 may be utilized to deliver the medical device 20. A grasper 62 may be employed through the working channel of the endoscope 60 to grasp the medical device 20 and traverse it through the patient's mouth and esophagus 12. Other means for grasping the medical device 20, such as clamps, forceps, or specially designed endcaps for the endoscope, can be used to deliver the medical device 20, as will be readily understood by those skilled in art. An overtube (not shown) or a wire guide may be utilized to facilitate passage of the endoscope 60 and medical device 20 through the esophagus 12 and into the gastric pouch 14.

The endoscope 60 may also be utilized to visualize the anastomosis 18 and guide placement of the medical device 20. Numerous other visualizations systems may be employed such as fiber-optic enabled catheter systems, fluoroscopy, ultra sound or similar visualization techniques. Generally, the medical device 20 is positioned such that the first anchor 30 is on a proximal side of the anastomosis 18 while the second anchor 32 is on a distal side of the anastomosis 18.

As shown in FIG. 3A, an inflation catheter 70 or other inflation device may be utilized alone or through the working channel of the endoscope 60 to engage the valves 48, 50 of the medical device 20 for inflation of the first and second expandable balloons 30, 32. The valves 48, 50 may take various forms as known in the art, such as one-way flap valves through which an inflation device may be passed. The inflation catheter 70 generally includes a needle 72 attached to a distal end thereof. One or more inflation catheters 70 may be attached to the valves 48, 50 prior to or after delivery of the medical device 20. The pusher 170 may also include suitable inflation passageways and connecting structures to both delivery and inflate the anchoring balloons 30, 32.

It will be recognized that once the medical device 20 is positioned as previously described, the expandable anchoring balloons 30, 32 may be operated and expanded into their deployed configurations, either sequentially or simultaneously. It may also be desirable to inflate the first expandable balloon 30 while leaving the second expandable balloon 32 in its delivery configuration, and then place the medical device 20 within the anastomosis 18. In this manner, the first expandable balloon 30 will prevent translation of the medical device 20 beyond the anastomosis 18 and help center the medical device 20. Removal of the medical device 20 may be accomplish by reversing the above-described steps, i.e., deflating the balloons 30, 32, and then grasping and retracting the medical device 20, such as with the aid of an endoscope and aforementioned tools.

It will also be recognized by those skilled in the art that the medical devices as described herein may be configured for permanent implantation or be made so that they are removable after a period of time. For example, the medical device 20 may be constructed of materials which are digestive-resistant, such as the plastics previously discussed, or metals such as stainless steel or nitinol. On the other hand, materials which are known to degrade and pass safely through the body may also be used. Semi-permanent implantations may be designed for later removal, and/or replacement. For example, when the medical device does not include a limiter, the entire device 20 may be periodically replaced with a new device having a tube with a differently sized tube lumen, to adjust the flow rate of stomach contents as needed by the patient. When the medical device 20 does include a limiter, the device may be removed to adjust the limiter, or the limiter may be adjusted while the medical device 20 remains in place within the anastomosis or other lumen. These and other variations of the medical device 20 and method of controlling the flow rate through an anastomosis will be readily apparent to those of ordinary skill in the art.

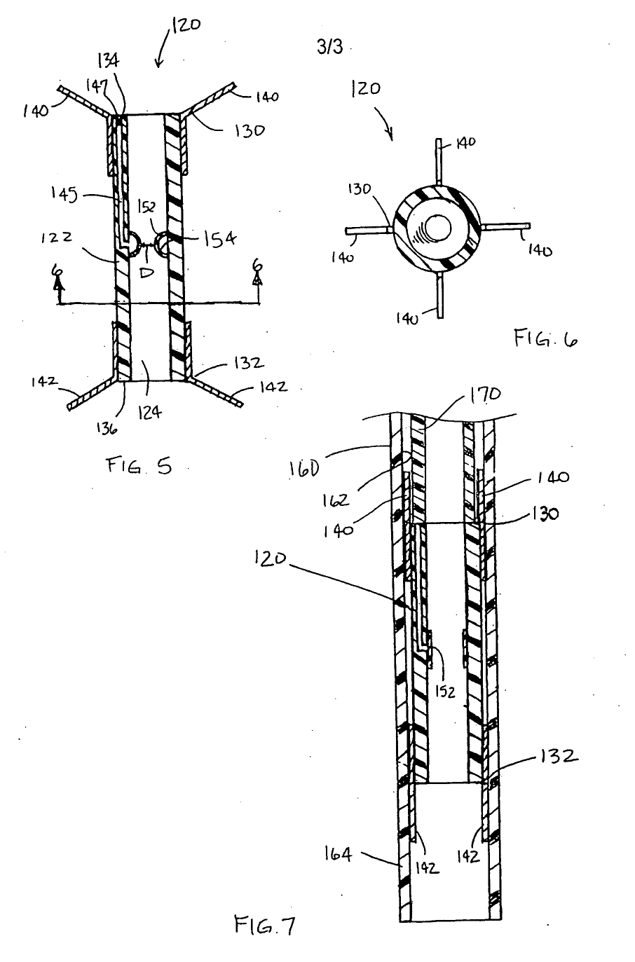

Turning now to FIGS. 5-7, another embodiment of a medical device 120 for controlling the contents through an opening is depicted in accordance with the teachings of the present invention. As with the prior embodiment, the medical device 120 includes a tube 122 defining the tube lumen 124 extending therethrough. A first radially extendable anchor 130 is divided at a proximal end 134 of the tube 122, while a second radially extendable anchor 132 is provided at a distal end 136 of the tube 122. In this embodiment, the first and second anchors 130, 132 each comprise a plurality of struts 140, 142. The plurality of struts 140, 142 are circumferentially spaced around the exterior of the tube 122, as best seen in FIG. 6. Although four struts 140 have been depicted in FIG. 6, any number of struts 140, 142 may be employed to form the first and second anchors 130, 132.

The plurality of struts 140, 142 may take many constructions and forms, but are generally operable between a deployed configuration as shown in FIG. 5, and a delivery configuration as shown in FIG. 7. The plurality of struts 140, 142 move radially outwardly between these configurations. In the deployed configuration, the struts 140, 142 have been shown as having a straight shape, although the struts 140, 142 may have a curved shape or other atraumatic design. In one construction, the plurality of struts 140, 142 are deflectable and biased towards the deployed configuration (FIG. 5). Here, the struts are preferably formed of a superelastic material such as nitinol, although other materials may be used including other metals, alloys or plastics. In another construction, the plurality of struts 140, 142 are formed of shaped memory material, such as Nitinol or similar alloys, and constructed to transition from the delivery configuration (FIG. 7) to the deployed configuration (FIG. 5) at about body temperature. As is known in the art, Nitinol and similar shape memory alloys may be formed to transition between two or more configurations at given temperatures through controlling the properties of the material's austenitic and martensitic phases and their transition temperatures. Cross members or mesh or other elements may extend between the struts 140, 142.

In the embodiment of FIGS. 5-7, a limiter is also provided which takes the form of an expandable balloon 152. The balloon 152 includes an inflation chamber 154. The expandable balloon 152 and its chamber 154 may be inflated utilizing an appropriate fluid medium that can be injected through a passageway 145 formed in the tube 122. As in the previous embodiment, a valve 147 may be utilized to regulate the passage of fluid through the passageway 145 and into the balloon chamber 154. Accordingly, it will be recognized that the smallest inner diameter D of the tube lumen 124 is adjustable through inflation or deflation of the balloon 152.

Deployment of the medical device 120 in an opening such as the anastomosis 18 may generally be performed using the above-described methods. However, the medical device 120 may further comprise a delivery sheath 160, as shown in FIG. 7, to aid in deployment of the medical device 120. The delivery sheath 160 includes a delivery lumen 162 sized to receive the medical device 120 in its delivery configuration. Relative translation of the medical device 120 and delivery sheath 160 is used to pass the medical device 120 beyond the distal end 164 of the delivery sheath 160. This relative translation may be accomplished with a pusher 170 (shown as a polymeric tube, but can be a mandrel solid wire, braided or stranded wire, wire guide, with or without reinforcing strands, strips, coils). When the plurality of struts 140, 142 are biased to the deployed configuration (i.e. rather than being manually manipulated or temperature dependant), the delivery sheath 160 can be utilized to overcome the bias of the struts 140, 142 and maintain the medical device 120 in its delivery configuration.

Upon exiting the delivery sheath 160, the plurality of struts 140, 142 will move radially outwardly and take their deployed configuration. Accordingly, the methods for deploying the medical device 120 may include delivering the medical device 120 and delivery sheath 160 to a position within the anastomosis 18, and then maintaining the position of the medical device 120 (e.g. with the pusher 170) while retracting in a proximal direction the delivery sheath 160. As such, the second expandable anchor 132 will be first deployed, and then the first expandable anchor 130 will be deployed.

It will be recognized that once the medical device 120 has been placed within an anastomosis 18 or other opening, the medical professional retains the ability to control and regulate the flow of contents from the gastric pouch 14 to the small intestine 16 by use of the limiting balloon 152. As such, the balloon 152 may be adjusted and modified to meet the requirements of the individual while leaving the medical device 120 in place. As needed, an inflation device may be delivered to the medical device 120 (e.g. endoscopically or even laparoscopically) for unlimited adjustment of the expandable balloon 152 and the smallest diameter D of the tube lumen 124, thereby controlling the flow rate of the stomach contents.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A medical device for controlling the flow of contents through an opening in tissue, the medical device comprising:
a tube defining a lumen extending therethrough, the tube and lumen defining a longitudinal axis of the medical device, the tube having a proximal end and a distal end;
a first anchor attached proximate the proximal end of the tube, the first anchor being radially extendable;
a second anchor attached proximate the distal end of the tube, the second anchor being radially extendable;
the first and second anchors being spaced apart about 5 mm to about 15 mm; and
the medical device operable between a delivery configuration and a deployed configuration, the first and second anchors taking radially extended positions in the deployed configuration and radially retracted positions in the delivery configuration.

2. The medical device of claim 1, wherein the first and second radially extendable anchors are expandable balloons.

3. The medical device of claim 2, wherein the tube defines inflation passageways in communication with the expandable balloons.

4. The medical device of claim 3, wherein the inflation passageway includes valves in communication therewith.

5. The medical device of claim 1, wherein the first and second radially extendable anchors each comprise a plurality of struts.

6. The medical device of claim 5, wherein the struts are deflectable and biased towards the deployed configuration.

7. The medical device of claim 6, wherein the struts are formed of a superelastic material.

8. The medical device of claim 5, wherein the struts are formed of a shape memory material and structured to transition from the delivery configuration to the deployed configuration at about body temperature.

9. A medical device for controlling the flow of contents through an opening in tissue, the medical device comprising:
a tube defining a lumen extending therethrough, the tube and lumen defining a longitudinal axis of the medical device, the tube having a proximal end and a distal end;
a first anchor attached to a proximal portion of the tube, the first anchor being radially extendable;
a second anchor attached to a distal portion of the tube, the second anchor being radially extendable;
a limiter attached to the tube, the limiter operable for varying the diameter of a portion of the lumen; and
the medical device operable between a delivery configuration and a deployed configuration, the first and second anchors taking radially extended positions in the deployed configuration and radially retracted positions in the delivery configuration.

10. The medical device of claim 9, wherein the limiter is a band positioned about the exterior of the tube.

11. The medical device of claim 10, wherein the band is positioned between the first and second radially extending anchors.

12. The medical device of claim 10, wherein the band is elastic, and wherein the band is structured to deform the tube.

13. The medical device of claim 9, wherein the limiter is an expandable balloon.

14. The medical device of claim 13, wherein the tube defines an inflation passageway in communication with the expandable balloon.

15. The medical device of claim 9, wherein the first and second radially extendable anchors are positioned at the proximal and distal ends of the tube, respectively.

16. A method for controlling the flow of contents through an opening in tissue, the method comprising:
providing a medical device having a tube and first and second radially extendable anchors attached to the tube, the tube defining a lumen extending therethrough, the medical device operable between a delivery configuration and a deployed configuration, the first and second anchors moving radially outwardly between the delivery configuration and the deployed configuration;
delivering the medical device in its delivery configuration to a location proximate the opening;
positioning the medical device such that the first anchor is on a proximal side of the opening and the second anchor is on a distal side of the opening;
operating the medical device to move the first anchor radially outwardly to its deployed configuration; and
operating the medical device to move the second anchor radially outwardly to its deployed configuration.

17. The method of claim 16, wherein the first and second radially extendable anchors are expandable balloons, and wherein the steps of operating the medical device include inflating the balloons.

18. The method of claim 16, wherein the first and second radially extendable anchors each comprise a plurality of struts and the medical device further includes a delivery sheath enclosing the plurality of struts, and wherein the steps of operating the medical device includes translating the delivery sheath relative to the medical device to expose the plurality of struts forming the first and second radially extendable anchors.

19. The method of claim 16, wherein the step of operating the first radially expandable anchor to its deployed configuration is performed prior to the positioning step.

20. The method of claim 16, wherein the medical device further includes an expandable limiter, and wherein the method further includes the step of adjusting the limiter to vary the flow rate of contents through the tube.

21. The method of claim 20, wherein the step of adjusting the limiter is performed while the medical device is positioned within the opening.
